(19) United States
(12) Patent Application Publication
(10) Pub. No.: US 2022/0015719 A1
(43) Pub. Date: Jan. 20, 2022

(54) SENSOR FOR USE IN IMAGING APPLICATIONS

(71) Applicant: Oxford University Innovation Limited, Oxford (GB)

(72) Inventors: Jamie WARNER, Oxford (GB); Martin TWEEDIE, Oxford (GB); Stuart GILCHRIST, Oxford (GB); Sean SMART, Oxford (GB)

(21) Appl. No.: 17/295,127

(22) PCT Filed: Nov. 15, 2019
(86) PCT No.: PCT/GB2019/053256

§ 371 Date: May 19, 2021

(30) Foreign Application Priority Data
Nov. 19, 2018 (GB) 1818806.0

(51) Int. Cl. A61B 5/00; H01L 41/113; H01L 41/193; H01L 41/312; A61B 5/055; A61B 6/03; A61B 6/00; A61B 5/113; G01T 1/24

(52) U.S. Cl. CPC A61B 5/7292; H01L 41/1132; H01L 41/193; H01L 41/312; A61B 5/055; A61B 2562/12; A61B 6/037; A61B 6/541; A61B 5/113; G01T 1/24; A61B 2503/40; A61B 6/032

(57) ABSTRACT

Electromagnetically transparent conductive materials, in particular nanomaterials, are used in a sensor along with piezoelectric materials to detect the motion of a subject to provide respiratory and cardiac gating for imaging techniques such as MRI, CT scans and PET.

### FIELD OF THE INVENTION

The present invention relates to sensors for use in methods of imaging, methods of imaging using said sensors, and to imaging apparatuses comprising said sensors.

### BACKGROUND OF THE INVENTION

Many imaging techniques involve the irradiation and detection of electromagnetic radiation. In particular, many medical imaging techniques such as magnetic resonance imaging (MRI), computer tomography (CT) scans and positron emission tomography (PET) involve the irradiation and detection of electromagnetic radiation to provide an image of a subject. Magnetic resonance imaging uses strong magnetic fields, magnetic field gradients and radio waves to generate images of the organs in the body. CT scans involve using many X-ray measurements taken from different angles to produce cross-sectional images of specific areas of a scanned object. Positron emission tomography (PET) involves the detection of gamma ray radiation emitted from the body by a radioactive tracer molecule that has previously been administered to a subject.

The above techniques may be used in a clinical setting by medical professionals to provide an image of a patient that can then be used to aid in the diagnosis of a disease or injury. Additionally, the above techniques can be used in preclinical medical and biological research to provide images of small animals such as mice and rats. Magnetic resonance imaging of small rodents is becoming of increasing importance in preclinical medical research as MRI provides a powerful diagnostic tool to non-invasively assess anatomy with high spatial resolution and excellent soft tissue contrast.

In both clinical and preclinical applications of the imaging techniques discussed above, the respiratory motion of the subject being imaged (whether human or animal) has been found to degrade the image quality. The motion of the subject due to the subject's heartbeat may also degrade the image quality. Attempts to minimise the effect of motion on the quality of the image include sophisticated image processing techniques applied to the image once it has been obtained, and respiratory gating. Respiratory gating involves monitoring the subject's rate of breathing and respiratory motion whilst the imaging technique is being carried out, and only obtaining images of the subject between the subject's breaths such that no images are obtained whilst the subject is moving due to respiration.

Respiratory gating techniques involve the use of sensors to detect respiratory motion of the subject being imaged whilst the imaging process is going on. A piezoelectric respiratory monitor for in vivo NMR, McKibben et al., Magnetic Resonance In Medicine 27, 338-342 (1992) describes the sensor for detecting respiratory motion of a subject during MRI imaging and to using it to affect a method of respiratory gating. The sensor described in this paper involves the use of a piezoelectric material to detect the movement of the subject due to respiration. The current generated in the piezoelectric material by movement of the subject is transmitted via a metallic strip to a detector to indicate when the subject is moving. Images can then be obtained between breaths of the subject in a method of respiratory gating. However, the devices and techniques described in this paper are not suitable for use in MRI or CT scanners because the sensor contains a metal electrode which damages the quality of the MRI image. A further drawback associated with the techniques described in this paper is that the sensor is not optically transparent and so the subject cannot be viewed adequately by those carrying out the imaging technique. Accordingly, the sensors described in this paper have not found wide usage in the imaging of small animals in preclinical research.

Other techniques of respiratory gating are discussed in MRI compatible small animal monitoring and trigger system for whole body scanners, Herrmann et al., Z. Med. Phys. 24 (2014) 55-64. Techniques described in this paper use a sensor comprising a mechanical PVC pressure pad to detect respiratory motion of the subject being imaged. The pressure pad is a respiratory balloon which detects motion of the subject due to respiration. The apparatuses described in this paper are difficult to set up and require recalibration if the subject is moved to a different scanner. The respiratory balloon is also extremely sensitive to changes in environmental pressure such as doors being opened and closed. A further drawback associated with the techniques is that when the subject being imaged is a small rodent, it is necessary to hold the subject very tightly in place, for example by taping it down.

Accordingly, there is a continued need for methods of imaging and sensors for use in imaging applications. In particular, there is a continued need for methods of providing respiratory and cardiac gating in imaging techniques such as MRI and CT scans, such as those carried out on small animals in preclinical research.

### SUMMARY OF THE INVENTION

The present invention is based on the surprising finding that electromagnetically transparent conductive materials may be used in imaging techniques such as MRI, CT scans and PET. Electromagnetically transparent conductive materials can be used in a sensor along with piezoelectric materials to detect the motion of a subject and transmit a signal of said detected motion to a user carrying out the imaging technique. Electromagnetically transparent conductive materials have been found not to disrupt the quality of images in MRI and CT scans when used as part of a piezoelectric sensor. This is believed to be because the materials are electromagnetically transparent, and also, for certain materials, because the materials can be fabricated so thinly such that they do not interfere with the electromagnetic radiation used in the imaging techniques. Various electromagnetically transparent conductive nanomaterials (such as graphene) can also be fabricated so thinly when used in sensors that they are transparent to visible light. Accordingly, sensors can be fabricated such that the user carrying out the imaging techniques can see the subject whilst it is being imaged, which can be advantageous in certain situations. Electromagnetically transparent conductive materials have surprisingly been found to provide the above advantages whilst also being able to transmit an electric signal from a piezoelectric material (as an indication that the subject is moving) as well as metal electrodes. Accordingly, electromagnetically transparent conductive materials can be used to replace metal electrodes.

According to an aspect of the invention, there is provided a method of imaging a subject, wherein the method comprises:
(i) placing a sensor in contact with a subject, wherein the sensor comprises one or more piezoelectric materials and one or more electromagnetically transparent conductive materials; and
(ii) obtaining an image of a region of the subject with an imaging apparatus.

According to yet another aspect of the invention, there is provided a sensor for use in an imaging method, wherein the sensor comprises a film comprising one or more piezoelectric materials and a film comprising one or more electromagnetically transparent conductive materials applied to the upper and lower surfaces of the film comprising the one or more piezoelectric materials.

According to yet another aspect of the invention, there is provided an imaging system wherein the system comprises an imaging apparatus such as an MRI scanner, a CT scanner, or a PET scanner, and a sensor according to the present invention.

According to yet another aspect of the invention, there is provided the use of a sensor of the present invention or an imaging system of the present invention in an imaging method.

According to yet another aspect of the invention, there is provided a process for fabricating a sensor according to the present invention, wherein the process comprises:

(a) forming a graphene film by chemical vapour deposition (CVD) upon the surface of a substrate;

(b) applying a polymer scaffold film to the graphene film by spin coating;

(c) removing the polymer scaffold film and graphene film from the substrate via etching;

(d) applying the polymer scaffold film and graphene film to an upper surface of a film of piezoelectric material such that the graphene film is sandwiched between the film of piezoelectric material and the polymer scaffold film;

(e) repeating steps (a) to (e) so as to form and attach a second film of graphene to a lower surface of the film of piezoelectric material;

(f) removing the polymer scaffold films;

(g) combining the product of steps (a) to (f) with one or more additional components such as a pair of electrodes so as to fabricate a sensor;

(h) encapsulating the sensor in a polymer layer.

### BRIEF DESCRIPTION OF THE DRAWINGS

FIG. 1 shows SEM images of the graphene film before and after transfer to PVDF. a) and b) are images of the graphene film grown on copper at low and high magnification. c) and d) show SEM images of the graphene film after transfer to a PVDF substrate both at low and high magnification. e) and f) show Raman spectra of graphene transferred to e) silicon wafer with 300 nm oxide layer, and f) to PVDF film. In f), the characteristics peaks of graphene can be seen along with a PVDF background.

FIG. 2 shows a) a layer of graphene transferred to one side of the PVDF, b) a second layer of PMMA deposited on top of the first layer of PMMA once the first layer has dried, and a further layer of graphene and PMMA transferred to the other side of the PVDF, c) the film cut into strips with requisite dimensions and the PMMA removed, and d) electrode contacts fabricated and the device encapsulated in tape.

FIG. 3a shows a comparison of the typical trace of the pulsed breathing of a sedated mouse measured using a respiratory balloon and a sensor of the invention. Also plotted are the trigger and gating signals generated from the respiratory signal. FIGS. 3b and 3c show stills from a dynamic MRI scan. b) is without gating and c) is respiratory gated with the prototype graphene sensor of the invention, showing a cross section of the upper body. FIG. 3 d) shows a 3D CT image of a mouse with the region around the liver and lungs highlighted.

FIG. 4 is an equivalent circuit diagram for a piezoelectric sensor connected to a voltage divider.

FIG. 5 shows the effect of mounting geometry on the measured potential. (a) Image of the measurement setup. (b to f)—plots of measured potential: (b) clamped at each point along the length and driven at point 11, (c) clamped on the sensor at position=1 with end of sensor resting, (d) clamped over the electrodes with the end resting, (e) clamped over the electrodes resting at 5.5 cm and the end, and (f) clamped over the electrodes and resting at 3.5 and 7.5 cm and the end.

FIG. 6 shows a comparison with an existing silver contacted sensor and summary of maximum potentials in different geometries. (a) Potential produced by silver contacted sensor in sensor clamped geometry. (b) Bar chart summarising the maximum signal generated in each of the mounting positions shown in FIG. 6, with position of measurement indicated: clamped at each point along the length and driven at point 11, clamped at position one with end of sensor resting, clamped over the electrodes with the end resting, clamped over the electrodes resting at 5.5 cm and the end, and clamped over the electrodes and resting at 3.5 and 7.5 cm and the end.

FIG. 7 shows the effect of tension on the measured signal. (a to e). Plots showing the variation of signal intensity along the sensor length as tension is increased up to around 2 MPa. (f) Summary of the maximum intensities plotted against applied stress.

FIG. 8 shows the effect of reducing the area of Graphene/PVDF/Graphene in the sensor. (a) Schematic showing the reduction in width. (b to d) Plots of measured potential with strip width in the (b) end clamped, (c) one rib, and (d) two ribs geometry respectively, showing rapid decline with decreasing width.

FIG. 9 shows details of the cradle used to hold the animal. (a) Schematic of the cradle detailing key components. (b) Image of the cradle with animal in place, ready for insertion into the CT scanner.

FIG. 10 shows the effect of gating on a dynamic MRI scan. Four consecutive frames taken from an (a) ungated scan and (b) scan gated using the graphene transducer, compared to an average image of the four gated frames. (c) Mean pixel values over 20 frames for ungated and gated scans in the area of the liver showing increase in stability with gating, with the averaged region highlighted in inset MRI image, and location in body indicated in a 3D CT image.

FIG. 11 shows cross-sections taken from a high resolution scan with and without gating. (a) Ungated images show significant distortion and blurring, and at higher contrast significant artefacts outside the body of the animal are visible. (b) Gated images at equivalent contrast show a marked reduction in artefacts both inside and outside of the body. The position of all scans is indicated on a 3D CT image.

FIG. 12 shows cross-sectional CT images showing the artefacts from the silver contacted sensor. (a to b) Images containing only the graphene sensor beneath the body: (a) ungated, artefacts highlighted; and (b) gated. (c) Images containing both the graphene and silver sensors, with significant artefacts visible resulting from X-ray scattering from the silver contacted sensor. The position of each dimension is indicated on a 3D CT image.

### DETAILED DESCRIPTION OF THE INVENTION

The sensor of the present invention comprises one or more piezoelectric materials and one or more electromagnetically transparent conductive materials. The sensor of the invention detects the motion of the subject being imaged. As such, the sensor comprises a motion sensor.

Any suitable electromagnetically transparent conductive material can be used providing that it is transparent to electromagnetic radiation and sufficiently conductive so that it can transmit electric charge that has built up in the piezoelectric material. Typically, the electromagnetically transparent conductive material is also sufficiently strong and flexible such that the sensor can withstand mechanical flexure in use without being damaged.

Typically, the one or more electromagnetically transparent conductive materials comprise one or more electromagnetically transparent conductive nanomaterials. Examples of electromagnetically transparent conductive nanomaterials include a two-dimensional material, a one-dimensional material, a composite material comprising a two-dimensional material and one or more additional nanomaterials, or any combination thereof.

Examples of one dimensional materials that can be used include carbon nanotubes and metal nanowires. Suitably, these materials are in the form of a film such as a carbon nanotube film or metal nanowire film.

Examples of suitable two dimensional materials that can be used include graphene or niobium diselenide. Other examples of two dimensional nanomaterials include graphyne, borophene, germanene, silicene, stanene, phosphorene, and, metal films.

Preferably, the one or more electromagnetically transparent conductive nanomaterials comprise graphene or niobium diselenide. More preferably, the one or more electromagnetically transparent conductive nanomaterials comprise graphene. Graphene is particularly preferred since it has been found to provide electromagnetic transparency so as to not interfere with the electromagnetic radiation used in CT scanners and MRI scanners whilst also providing sufficient electrical conductivity to transmit electric charge from the piezoelectric material so as to transmit a signal of the motion of the subject being imaged. This is, in part, because graphene can be fabricated so thinly that it does not interfere with electromagnetic radiation whilst maintaining its electrically conductive properties. Other advantages associated with graphene are that it is optically transparent, very strong, flexible, stable, and can be grown over larger areas than other electromagnetically transparent conductive nanomaterials.

The one or more electromagnetically transparent conductive nanomaterials may comprise a composite material comprising a two-dimensional material and one or more additional nanomaterials. The two dimensional material in the composite may be any of the two dimensional materials discussed above. The additional nanomaterial in the composite may be any of the one dimensional materials discussed above such as carbon nanotubes or metal nanowires. The additional nanomaterial may also comprise other nanomaterials such as fullerenes.

The one or more electromagnetically transparent conductive nanomaterials may comprise any one or combination of the materials discussed above. Preferably, the one or more electromagnetically transparent conductive nanomaterial comprise graphene, and more preferably consist exclusively of graphene.

In other embodiments, the one or more electromagnetically transparent conductive materials comprise one or more conductive polymers. Examples of conductive polymers that may be used include comprise polyaniline, polyindole, polypyrrole, poly(3, 4-ethylenedioxythiophene) polystyrene sulfonate (PEDOT:PSS), or a combination thereof.

In other embodiments, the one or more electromagnetically transparent conductive materials comprise a film of electromagnetically transparent conductive particles, such as electromagnetically transparent conductive microparticles. Examples of particles and microparticles include graphite, carbon black, or a combination thereof. In some embodiments, the one or more electromagnetically transparent conductive materials comprise a film of electromagnetically transparent conductive particles, such as electromagnetically transparent conductive microparticles, and also one or more of the electromagnetically transparent conductive nanomaterials discussed above.

In other embodiments, the one or more electromagnetically transparent conductive materials comprise a composite film. The composite film may typically comprise one or more electromagnetically transparent conductive nanomaterials, electromagnetically transparent conductive particles such as microparticles, or a combination thereof; and a polymer binder. The one or more electromagnetically transparent conductive nanomaterials, and the electromagnetically transparent conductive particles may be any of those discussed above. The polymer binder may be any suitable polymeric binder known to the skilled person. Examples of polymer binders that may be used in the composite films include polyaniline, polyindole, polypyrrole, poly(3, 4-ethylenedioxythiophene) polystyrene sulfonate (PEDOT:PSS), poly(methyl methacrylate), polystyrene, polycarbonate, acrylonitrile butadiene styrene, polyethylene, polypropylene, polyurethane, poly(lactic acid), poly(vinyl chloride), epoxy resins, polyamide, or a combination thereof.

In other embodiments, the one or more electromagnetically transparent conductive materials comprise a film deposited from a liquid suspension of particles of the one or more electromagnetically transparent conductive materials. Typically, the one or more electromagnetically transparent conductive materials comprise graphene, graphite, or a combination thereof, although it will be understood that any of the electromagnetically conductive materials discussed above may also be used and deposited from a liquid suspension so as to form a film.

Examples of liquid suspensions that can be used to deposit a layer of electromagnetically transparent conductive materials include commercially available graphene paints, and similar such formulations. Such formulations typically contain flakes of nanomaterial such as 2D nanomaterials such as graphene. Graphene flakes may be synthesised by any suitable method, as discussed in further detail below. In liquid suspensions of graphene flakes, said graphene flakes are typically prepared by exfoliation. The liquid suspensions may thus comprise graphene particles of only a few layers in thickness. Such particles would be graphene nanoparticles. However, in said suspensions, graphene nanoparticles comprising only a few layers of graphene may sometimes agglomerate to form many layered graphene particles. When agglomerated, such particles may be larger than 1 micron in size, and contain many layers of graphene stacked upon one another. The term graphene as used herein is intended to cover said particles.

It will be understood that the one or more electromagnetically transparent conductive materials must be compatible with the one or more piezoelectric materials discussed in further detail below. For example, it will be appreciated that some of the piezoelectric materials discussed below, such as the polymer PVDF, cannot be processed at temperatures higher than around 70° C. If an electromagnetically transparent conductive material requires processing at temperatures higher than 70° C., then it would not be compatible with certain piezoelectric materials. Similarly, in the case where the one or more electromagnetically transparent conductive materials are applied using a suspension, certain liquids may be incompatible with certain piezoelectric materials in that the liquids may degrade them. It will be appreciated by those skilled in the art which liquids would be incompatible with certain piezoelectric materials, and which piezoelectric materials cannot be processed above certain temperatures. Additionally, where the one or more electromagnetically transparent conductive materials comprise a polymer such as a polymer binder or a conductive polymer, it will be appreciated by those skilled in the art that certain polymers may not be compatible with certain piezoelectric polymers, due to the possibility of the different polymeric materials mixing at the interface between the materials. Accordingly, the one or more electromagnetically transparent conductive materials are selected so as to be compatible with the one or more piezoelectric materials that are used.

The one or more piezoelectric materials can be any suitable piezoelectric material that can generate electric charge as a result of the pressure applied by the motion of the subject being imaged (whether pressure due to cardiac motion or respiratory motion). Typically, the one or more piezoelectric materials comprise one or more piezoelectric polymers, one or more piezoelectric polymer-composite materials, or one or more composite materials comprising one or more polymers and one or more piezoelectric ceramic materials, or combinations thereof.

Preferably, the one or more piezoelectric materials comprise one or more piezoelectric polymers. More preferably, the one or more piezoelectric materials comprise one or more piezoelectric fluorinated polymers. Examples of piezoelectric fluorinated polymers include polyvinylidene fluoride (PVDF), a copolymer of polyvinylidene fluoride (PVDF), or a combination thereof. Most preferably, the piezoelectric material comprises polyvinylidene fluoride (PVDF). Examples of copolymers of polyvinylidene fluoride (PVDF) include polyvinylidene fluoride-trifluoroethylene-chlorotrifluoroethylene [P(VDF-TrFE-CTFE)] and polyvinylidene fluoride-trifluoroethylene-chlorofluoroethylene [P(VDF-TrFE-CFE)]. The piezoelectric polymer may comprise any one or more of the materials described above.

Alternatively, the piezoelectric material comprises one or more piezoelectric polymer-composite materials. The piezoelectric polymer in these materials is typically one of the piezoelectric polymers discussed above. The piezoelectric polymer-composite materials typically also comprise one or more nanomaterials present in the polymer composite. Examples of such nanomaterials include graphene, carbon nanotubes, fullerenes, or any combination thereof.

Alternatively, the piezoelectric material comprises one or more composite materials comprising one or more polymers and one or more piezoelectric ceramic materials. Said composite materials typically comprise a polymer selected from polyvinylidene fluoride (PVDF), polydimethylsiloxane, epoxy resin, polyurethane, or a combination thereof. Said composite materials also comprise a piezoelectric ceramic material. Examples of such materials include a ceramic selected from lead zirconate titanate, (PZT), lanthanum-modified lead zirconate titanate (PLZT), quartz, lithium niobate, or a combination thereof.

In preferable embodiments, the sensor comprises a piezoelectric polymer such as polyvinylidene fluoride (PVDF) and graphene as the electromagnetically transparent conductive material.

The graphene for use in the sensor can be synthesised in any known to way to make graphene. Examples of such techniques include exfoliation, hydrothermal self-assembly, epitaxy, and carbon nanotube slicing. Preferably, the graphene is synthesised via epitaxy and more preferably by chemical vapour deposition (CVD).

Chemical vapour deposition is particularly preferred since it has been found that when this technique is used to provide the graphene component of the sensor and the sensor is used in imaging techniques, the sensor performance is more stable than when graphene is derived by other processes such as the deposition of a film from a liquid phase exfoliated suspension. In addition to this, the graphene formed from chemical vapour deposition has been found to be better at transmitting the electric current from the piezoelectric material than graphene formed by other techniques such as exfoliation. Accordingly, in a particularly preferred embodiment, the graphene is formed by chemical vapour deposition.

Preferably, the sensor comprises a film of piezoelectric polymer with a film of the one or more electromagnetically transparent conductive materials applied to the upper and lower surfaces of the piezoelectric polymer film. Preferably, the piezoelectric polymer film comprises PVDF and the one or more electromagnetically transparent conductive materials comprises graphene.

The piezoelectric polymer film can be any suitable thickness sufficient for detecting the motion of the subject being imaged by generating sufficient charge within the film. Typically, the piezoelectric polymer film is from 9 μm to 750 μm in thickness, preferably from 50 μm to 500 μm in thickness, more preferably from 50 μm to 150 μm in thickness, and most preferably from 100 μm to 120 μm in thickness, although the skilled person will understand that other film thicknesses outside of these limits may work just as well.

The graphene films are typically from 1 to 100 layers of graphene in thickness, and preferably from 1 to 10 graphene layers in thickness. Preferably, each graphene film comprises a monolayer of graphene (1 layer). However, as discussed above, where the one or more electromagnetically transparent conductive materials comprise a film deposited from a liquid suspension, exfoliated graphene particles present in the film may comprise many more layers of graphene stacked upon one another.

The sensor may further comprise a film of polymer applied on top of each graphene film. In this respect the piezoelectric polymer is sandwiched between two layers of graphene. The two layers of graphene are then sandwiched between two layers of the additional polymer. The additional polymer may serve to protect the graphene and piezoelectric material from the outside environment. Typically, the polymer comprises polyethylene terephthalate (PET), polypropylene or polyethylene, and more preferably polypropylene or polyethylene terephthalate (PET).

The sensor may be constructed of any suitable dimensions for the imaging job at hand. For example, a sensor that is longer and wider may be constructed for a larger subject to be imaged such as a human. In contrast, a much smaller sensor may be constructed for imaging s maker subject such as a rodent.

When used to image small animals such as mouse or other rodent, the sensor is typically from 5 mm to 50 mm in width, and more preferably from 5 mm to 20 mm in width. The sensor is typically from 50 mm to 500 mm in length, and more preferably from 100 to 150 mm in length.

The sensors of the invention typically comprise a pair of electrodes that are electrically coupled to the one or more electromagnetically transparent conductive materials. These electrodes are for transmitting the electric current from the electromagnetically transparent conductive material such that it can be detected by an operator of the imaging method. Typically, the electrodes are fabricated form metal. If fabricated from metal, the electrodes are positioned in the sensor such that they do not interfere with the image of the region of the subject being obtained. This is in contrast to piezoelectric sensor devices known in the art that comprise a piezoelectric material and a conducting metal strip to conduct the electric charge away from the piezoelectric material. In these devices, the metal strip is positioned within the sensor directly adjacent to a region of a subject that an image is being obtained of such that the metal strip damages the quality of the image being obtained.

The sensors of the present invention may be fabricated using suitable methods known in the art for forming a polymer film and providing a film of one or more electromagnetically transparent conductive materials upon the surfaces of the film.

Preferably, the sensors of the invention are formed by the process of the invention. Thus, according to an aspect of the invention, there is provided a process for fabricating a sensor of the invention, wherein the process comprises:

(a) forming a graphene film by chemical vapour deposition (CVD) upon the surface of a substrate;

(b) applying a polymer scaffold film to the graphene film by spin coating;

(c) removing the polymer scaffold film and graphene film from the substrate via etching;

(d) applying the polymer scaffold film and graphene film to an upper surface of a film of piezoelectric material such that the graphene film is sandwiched between the film of piezoelectric material and the polymer scaffold film;

(e) repeating steps (a) to (e) so as to form and attach a second film of graphene to a lower surface of the film of piezoelectric material;

(f) removing the polymer scaffold films;

(g) combining the product of steps (a) to (f) with one or more additional components such as a pair of electrodes so as to fabricate a sensor;

(h) encapsulating the sensor in a polymer layer.

Preferably, the polymer scaffold film comprises poly (methyl methacrylate) (PMMA).

Preferably, step (d) further comprises applying an additional film of polymer scaffold to the polymer scaffold film. This step improves the quality of the transferred graphene film as well as sealing the edges, thereby preventing delamination of the first layer of graphene during transfer of the second layer of graphene.

Preferably, wherein the chemical vapour deposition in step (a) comprises growing a graphene monolayer upon a copper foil substrate.

The sensors of the invention are used in methods of the present invention which comprise methods of imaging a subject comprising:

(i) placing a sensor in contact with a region of a subject, wherein the sensor comprises one or more piezoelectric materials and one or more electromagnetically transparent conductive materials; and

(ii) obtaining an image of the region of the subject with an imaging apparatus.

The imaging method can be any imaging method. Preferably, the imaging method is one that requires detecting movement of the subject being imaged. Examples of imaging methods include a magnetic resonance imaging (MRI) method, a computed tomography (CT) method, or a positron-emission tomography (PET) method. Preferably, the method of the invention is a magnetic resonance imaging (MRI) method or a computed tomography (CT) method. Most preferably, the method of the invention is a magnetic resonance imaging (MRI) method.

The region of the subject being imaged may comprise the entirety of the subject (i.e. the entirety of a subject's body is being imaged). Alternatively, the region being imaged may be a region of the subject that is not the entirety of the subject (for example a specific body part).

The method of the invention may be a medical imaging method in a clinical setting in which a human subject has an image taken of them. In such methods, said image may help a medical professional to arrive at a diagnosis of a disease or condition of the patient.

Alternatively, the method of the invention may be a method performed in a preclinical setting such as in medical research performed upon animals.

It is envisaged that the methods and sensors of the present invention will find particular utility in methods performed in a preclinical setting such as those performed on animals.

The subject may therefore be a mammalian subject, such as a human subject. Alternatively, the subject may be an animal. Preferably, the subject is a mammalian subject of the order Rodentia (a rodent), and more preferably a mouse or a rat.

The method of imaging of the invention typically comprises irradiating the subject or a region of the subject with electromagnetic radiation so as to provide an image of the subject or region of the subject.

The method may be an MRI imaging method that comprises using an MRI apparatus to irradiate the subject or a region of the subject with electromagnetic radiation so as to provide an image of the subject or region of the subject. Alternatively, the method may be an CT scanning method that comprises using a CT scanner apparatus to irradiate the subject or a region of the subject with electromagnetic radiation so as to provide an image of the subject or region of the subject.

The method may further comprise detecting movement of the subject with the sensor. In this regard, the movement of the subject causes pressure to be applied to the piezoelectric material within the sensor which causes a build-up of charge within the piezoelectric material. The conductive material such as graphene then conducts the charge such that the charge flows as current through the material to the sensor's electrodes where the current is detected as an indication of movement of the subject.

The method preferably comprises detecting the movement of the subject with the sensor, wherein the movement of the subject is movement associated with the breathing of the subject or the heartbeat of the subject. More preferably, the method comprises detecting the motion of the subject due to respiration and heartbeat of the sensor.

The method typically further comprises detecting movement of the subject with the sensor, wherein the movement of the subject is movement associated with the breathing of the subject or the heartbeat of the subject; and obtaining the image of the region of the subject at a time interval between breaths or heartbeats of the subject. Such a process is known in the art as gating. Gating involves only obtaining images of a subject when the subject is not moving due to respiratory motion, cardiac motion or otherwise. Gating can thus provide an image of the region of the subject where the disruptive effects of the movement of the subject are minimised so as to produce an improved image. Methods of the invention may thus aid in providing an improved and more efficient diagnosis of a disease in a patient, or quicker and easier conclusion being reached in medical research where the method involves imaging an animal as part of preclinical research.

Surprisingly, sensors of the invention have been found to be superior to motion sensors for use with MRI known in the art with regard to detecting motion of subjects. In tests, it has been found that sensors of the invention can detect both respiratory and cardiac motion of a subject, whereas sensors known in the art such as those comprising a pneumatic balloon only detect respiratory motion and do not detect cardiac motion.

According to another aspect of the invention, there is provided an imaging system, wherein the system comprises an imaging apparatus such as an MRI scanner, a CT scanner, or a PET scanner, and a sensor according to the present invention.

Methods of imaging of the invention, along with imaging systems of the invention are in accordance with those known in the art with the exception that the methods and systems employ a sensor of the invention. Accordingly, the specific additional details of apparatus such as MRI machines and CT scanners, methods of operating said apparatus and carrying out said methods, and methods of processing images obtained from said methods do not need further description.

### EXAMPLES

### Example 1—Preparation of Graphene Films

Graphene films were prepared by chemical vapour deposition with the following process steps.

1. A 25 μm thick piece of copper foil was mechanically polished by cloth wheel and two types of abrasive polish (1^{st} lustre, 2^{nd} rouge), both purchased from RS Components.

2. The copper foil was cleaned of polishing residue by wiping with ethanol.

3. The copper foil was cut to a size of 7×15 cm.

4. The copper foil was cleaned by ultrasonication for 10 minutes in each of 1 M hydrochloric acid, deionised (DI) water, pure deionised water, acetone, and isopropanol.

5. The foil was then dried by a nitrogen gun, placed on an alumina crucible, and sealed in the centre of a four inch quartz tube in a tube furnace.

6. The quartz tube was sealed and purged with hydrogen gas diluted in argon.

7. Gas flow rates were reduced and the furnace temperature was increased to 1060° C.

8. The foil was then annealed for 1 hour once furnace reached 1060° C.

9. Methane was introduced to the gas mixture flowing through the furnace for one hour.

10. Methane flow was then stopped, the furnace switched off, and the foil removed from the centre of the furnace to facilitate rapid cooling.

11. Once furnace was at room temperature, the foil with graphene as a product formed upon its surface was removed from the furnace.

The exact parameters of the process described above are shown in table 1 below.

TABLE 1

| | Flow Rate (sccm) | | | | |
|---|---|---|---|---|---|
| Step | Ar | H₂ | CH₄ | Temperature (° C.) | Time (min.) |
| Purge | 2000 | 500 | 100 | RT | 5 |
| | 2000 | 500 | 0 | | 30 |
| Anneal | 500 | 100 | 0 | RT → 1060 | 90 |
| | 500 | 100 | 0 | 1060 | 60 |
| Growth | 500 | 100 | 5-10 | | 60 |
| Cooling | 500 | 100 | 0 | 1060 → RT | 120 |

### Example 2—Spin Coating of Graphene Films

The following steps were performed on graphene films prepared as in example 1.

1. The graphene on a copper foil substrate prepared in example 1 was placed in a spin coater and fixed to the spin coater chuck by vacuum.

2. A spin coating program consisting of 5 seconds at 500 rpm followed by 60 seconds at 4000 rpm was started.

3. During 5 seconds at 500 rpm, PMMA (8 wt % in anisole, molecular weight=495K) was deposited onto the surface of the graphene. This process is known as dynamic dispense.

4. Spin coating was allowed to proceed and the sample removed once program had completed.

### Example 3—Etching of Graphene Films from Copper Substrate

The following steps were performed on spin coated graphene films prepared as in Example 2.

1. The PMMA coated graphene film was dried of remaining anisole by baking the coated copper foil on a hotplate at 180° C. for 90 seconds.

2. Graphene was removed from the back side of the copper foil by placing the PMMA side down on aluminium plate and etching in 90 W oxygen plasma for 2 minutes.

3. Copper was then etched by floating the PMMA/graphene coated copper foil on a solution of 0.1 M (NH₄)S₂O₈ in deionised water. The solution was then refreshed once by pumping out the old solution and replacing with clean solution.

4. The floating PMMA/graphene film was washed by pumping out the etchant and exchanging with deionised water. This process was repeated 3 times, allowing 30 minutes between each exchange.

### Example 4—Transfer of Etched Graphene Film to Piezoelectric Polymer Film

The following steps were carried out on etched graphene films coated with a PMMA polymer scaffold prepared as in Example 3 above.

1. A 110 μm thick PVDF film was cut to a size of 7.5×16 cm (length and width).

2. The PVDF film was sonicated in acetone followed by isopropanol for 10 minutes each, and solvent residues removed by placing it in a vacuum desiccator for 30 minutes.

3. Graphene was transferred by manually scooping it from the deionised water onto the centre of one side of the PVDF. This film was left to dry at room temperature for 2 days at room temperature and pressure.

4. The dried film was spincoated with an additional layer of PMMA (1 wt % in anisole, molecular weight=495K; same parameters as before), and allowed to dry. This helps the graphene to conform to the substrate and prevented delamination during the second transfer.

5. During this time a second piece of graphene was prepared as described above in examples 1 to 3.

6. This second piece was of graphene was then transferred onto the other side of the PVDF as described above, aligned with the first layer.

7. The second layer of graphene was once again allowed to dry for 2 days at room temperature and pressure.

8. PMMA (1 wt % in anisole, molecular weight 495K) was then drop cast onto the second layer of graphene and allowed to dry.

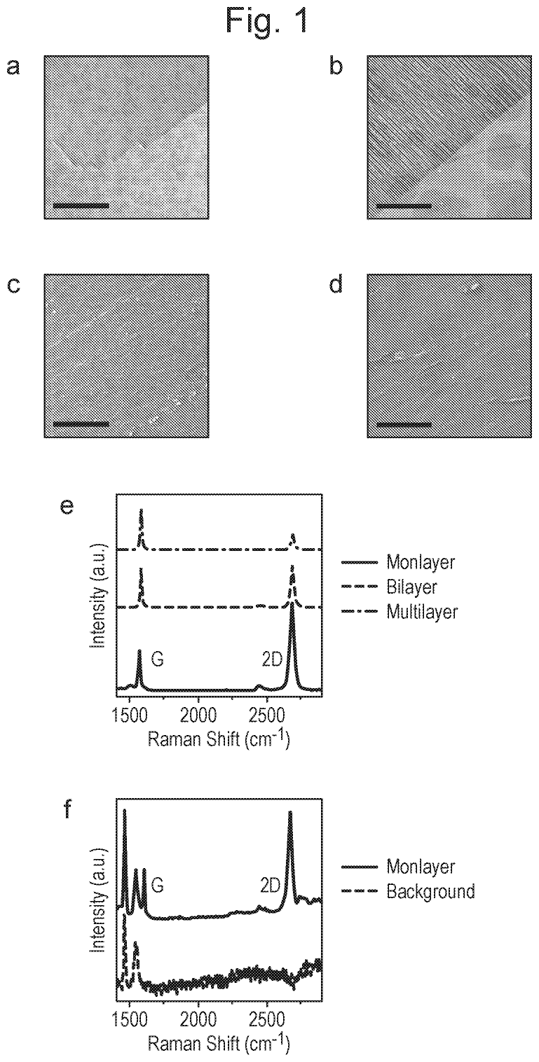

FIG. 1 shows SEM images of the graphene film before and after transfer to PVDF. a) and b) are images of the graphene film grown on copper at low and high magnification. c) and d) show SEM images of the graphene film after transfer to a PVDF substrate both at low and high magnification. e) and f) show Raman spectra of graphene transferred to e) silicon wafer with 300 nm oxide layer, and f) to PVDF film. In f), the characteristics peaks of graphene can be seen along with a PVDF background.

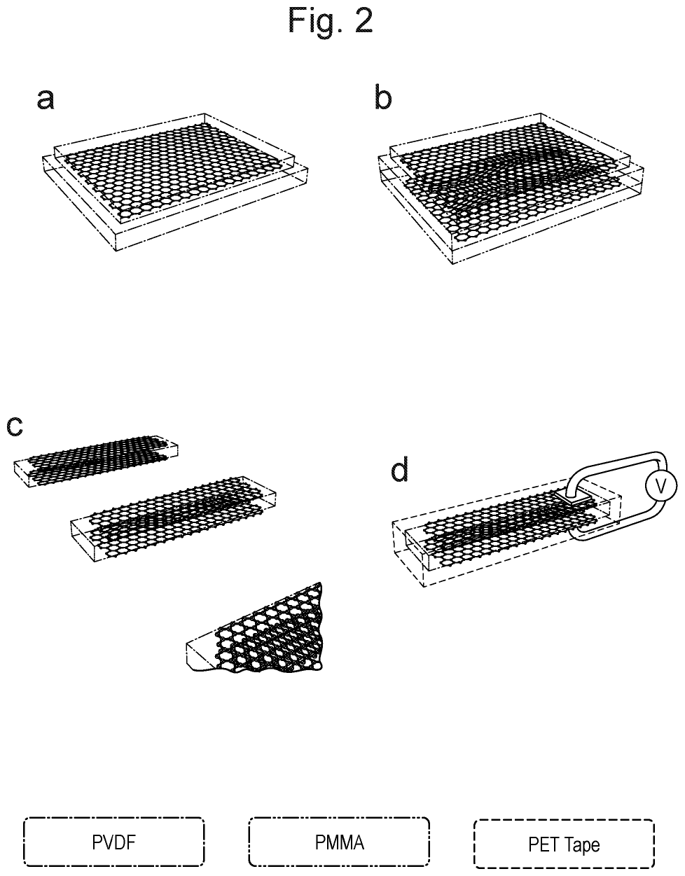

FIG. 2 shows a) a layer of graphene transferred to one side of the PVDF, b) a second layer of PMMA deposited on top of the first layer of PMMA once the first layer has dried, and a further layer of graphene and PMMA transferred to the other side of the PVDF, c) the film cut into strips with requisite dimensions and the PMMA removed, and d) electrode contacts fabricated and the device encapsulated in tape.

### Example 5—Sensor Fabrication

The piezoelectric polymer film with a graphene film adhered to both its upper and lower surface prepared as in example 4 had the following steps performed on it:

1. 4 mm strips were removed from each edge of the PMMA/graphene/PVDF/graphene/PMMA composite film to remove regions of the film that may have been damaged during transfer.

2. Six 12 mm×140 mm strips were cut from the remaining material

3. PMMA was removed from the strips by immersing them in acetone for 48 hours at room temperature.

4. Electrodes were made by creating a frame of carbon tape (5 mm×5 mm with a 3 mm×3 mm window) and silver paint was used to affix a section of multicore wire to one side.

5. These electrodes were affixed (one to each side, at one end of the sensor 10-20 mm from the end) using silver paint.

6. Once the silver paint was dry, the electrodes were encapsulated in epoxy resin.

7. Once the epoxy resin was cured, the sensors were cut down to the required length of 120 mm.

8. The sensors were encapsulated in 65 μm thick polypropylene tape on each side. The tape was cut to the required size (initially 15 mm but later customised to fit cradle (see example 6 below)).

9. The electrodes were further supported by covering them in 19 mm wide PVC insulation tape.

10. To minimise induction in the wires they were tightly twisted together to form a twisted pair.

### Example 6—Testing of Prototype Sensors

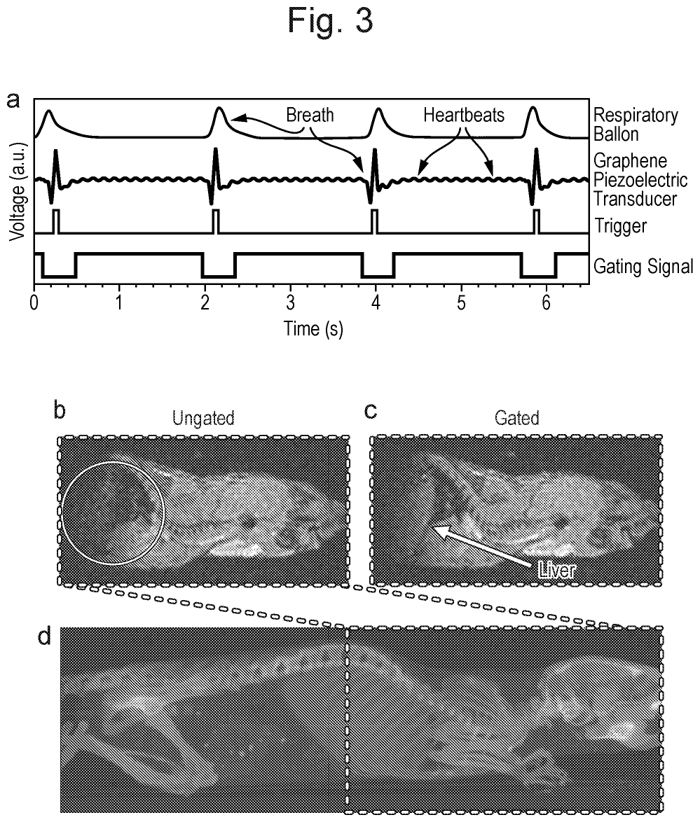

Small scale prototype devices (10 to 50 mm) were fabricated using the above methods, to test the efficacy of graphene in this application. Facile mounting of the sensor device by simply placing it in contact with the body when it was loaded led to very effective monitoring of the respiratory cycle and, surprisingly, also signals from the cardiac cycle. Data from this early prototype device as well as from a respiratory balloon monitoring the same subject are plotted in FIG. 3. Note the faster rise and fall times produced by the sensor of the invention, as well as the presence of cardiac signals that are absent in the respiratory balloon trace. Also plotted is an example of the trigger produced from the peak of the respiratory signal that is subsequently converted into a gating signal, with acquisition turned on during the peaks and off during the troughs, when the body is in motion. Basic scans were then performed to prove the functionality of the sensor device. In the ungated image (FIG. 3b), respiration causes distortion of the image, most prominently causing the liver to periodically move out of frame. With gating, the image is stable (FIG. 3c). FIG. 3d shows a CT image of the mouse with the region where the most disturbance occurs around the liver and lungs highlighted. Having verified the working principle and MRI compatibility, a sensor was designed that could be integrated into a custom 3D printed cradle with built in carbon-fibre based heating and ECG measurement technology.

### Example 7—Set Up of the Sensor Prior to Testing, and Preliminary Tests

The sensor fabricated as in example 5 was tested so as to quantify its behaviour and determine the optimal mounting geometry. The respiratory signal was simulated using a custom built actuator using a small cantilever arm mounted to a DC motor and controlled by a timing circuit (RS Components) to apply force to the sensor. The resultant signals were measured using a Biopac MP150 unit with DA100C amplifier. The MP150 provides high measurement rates and signal to noise ratio, enabling accurate measurement of the generated potential. The devices were connected to a shielded potential divider to reduce electrical noise and enable adjustable control of the input signal intensity, and the potential between the two sides as a result of small mechanical deformations measured at a rate of 1 kHz. The reduction of electrical noise was critical here due to the high amplification used. By enclosing all components up to the sensor and using twisted pair leads, the noise was reduced by several orders of magnitude. Measurements were recorded using the AcqKnowledge® software package. The resultant signals required conditioning as they were found to exceed the limits of the detector. The signal magnitude was reduced by means of a resistive voltage divider of appropriate transfer function (equation 1), in which the input signal is applied across a pair of resistors and the output taken across the second.

$${H}_{div} = \frac{{R}_{2}}{{R}_{1} + {R}_{2}} \qquad Equation 1$$

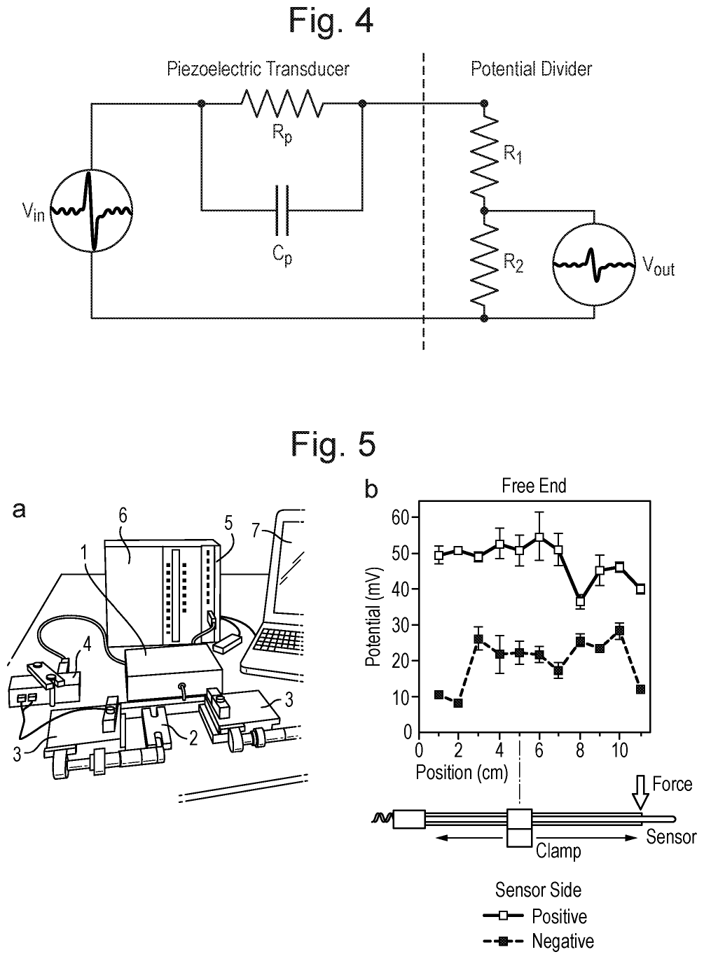

Calibration of the potential divider was necessary due to the transient signal generated by the sensor resulting in deviation from Equation 1. This can be understood with reference to the equivalent circuit shown in FIG. 4. The transducer itself contains a series resistance and capacitance which also results in signal decay in static or direct current applications. Rather than attempt to measure these and calculate H directly, we chose instead to estimate it by applying a very small mechanical signal and comparing the amplitude of divided and undivided (i.e. R1=0 and R2=1) signals. We achieved sufficient signal reduction with R1=39 kW and R2=12 kW, leading to H=0.147±0.003 (cf. H=0.235 given by Equation 1).

The high level of amplification resulted in significant detection of 50 Hz mains hum, so the entire assembly was shielded, and a twisted pair lead used to connect the sensor to the divider. We further retrospectively subtracted a fitted 50 Hz sine function from the measured potential, preserving the signal amplitude in contrast to low pass or Butterworth notch filtering, which led to significant signal attenuation.

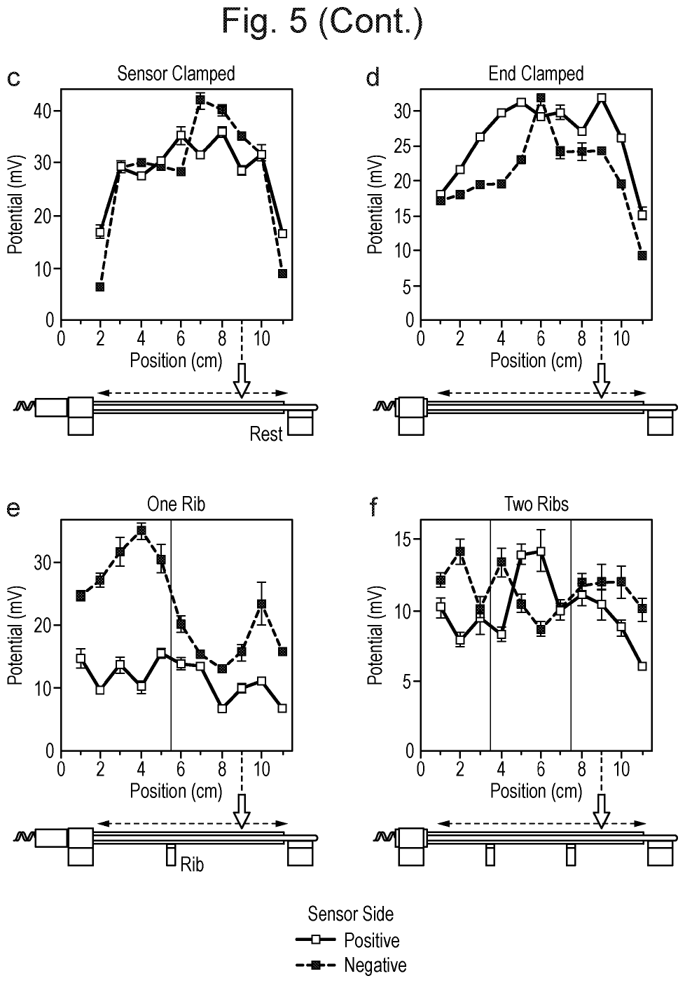

The sensor was mounted in a range of configurations and force was applied along its length, to both the negative and positive sides of the PVDF (as defined by the manufacturer). The maximum applied force as given by the stall torque of the motor at 6 V was 193 mN, and the maximum detection of the cantilever arm in the absence of physical resistance was 5 mm. An image of the measurement setup is shown in FIG. 5a: a mechanical signal (1) stimulates the sensor (2), which is fixed to two linear translation stages (3), producing a signal that is reduced by the potential divider (4) before being amplified (5) and measured (6), a read out of which is recorded and displayed on a connected PC (7). The measured potentials and illustrations of the various mounting geometries are displayed in FIGS. 5b to 5f.

In general, the signal intensity was found to be similar on both sides of the sensor, with notable exceptions in the free end and one rib geometries. These differences can be explained by the convex curvature along the length of the positive side (and therefore concave curvature of the negative side, analogous to a retractable tape measure). In both cases, the curvature of the film enables easy deflection on the positive side, while resisting deformation on the negative side: in the free end geometry (FIG. 5b), the cantilever type deflection is easier on the positive side, and in the one rib geometry (FIG. 5e), the 3-point bending is easier on the negative side. In the two ribs geometry (FIG. 5f), the magnitude of deflection of the sensor is more strongly inhibited by the supporting ribs, leading to similar responses on both sides, and reduced overall magnitude.

The maximum signal amplitude was achieved by free end mounting (FIG. 5b). However, since the body of the animal needs to be supported this would be difficult to practically achieve in the final cradle, and we settled instead on the inclusion of ribs, similar to the geometry depicted in FIG. 5f. While this produced the lowest signal amplitude, this geometry has the advantages of ease of mounting and animal placement, and the stability of the signal along the length of the sensor ensuring a high degree of consistency in spite of inevitable variations in the body position.

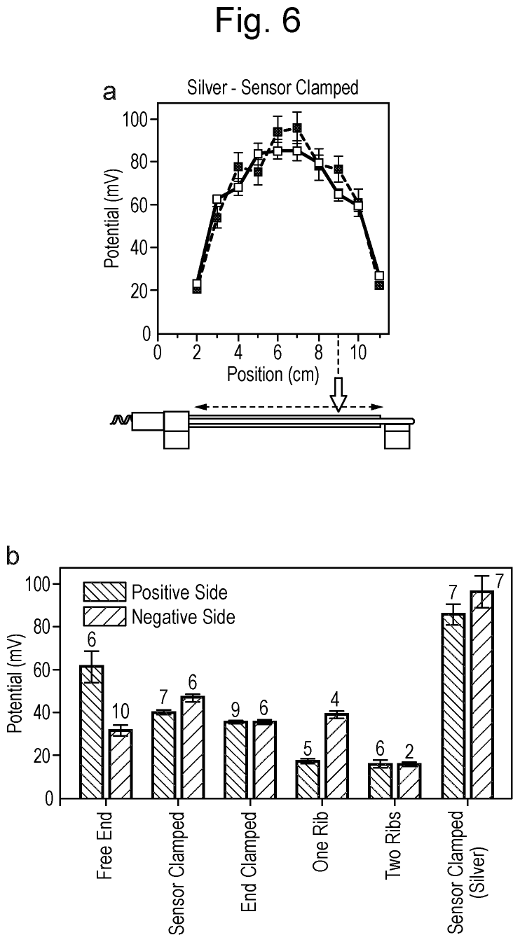

We also tested the response of a silver-contacted sensor, mounted in the sensor clamped geometry (FIG. 6a). The behaviour was similar to that of our graphene contacted device, with the larger signal intensity a result of the greater widths of the strip (18 mm vs. 12 mm) and lower resistance of the comparatively thick silver film. The maximum signal magnitude and position for all geometries and both sides are summarised in FIG. 6b. We also plot the potential measured by the silver contacted sensor for comparison.

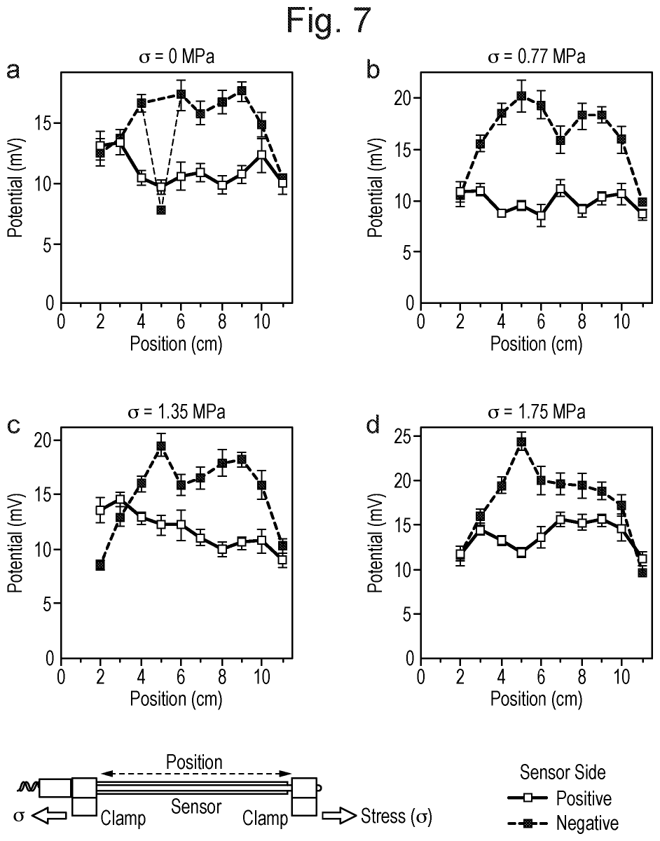

The study on the effects of mounting geometry by studying the effect of tensile stress on the generated voltage was continued. The sensor was clamped as in FIG. 5c, and a second clamp placed at the sensor end. A tensile strain (up to approximately 2 MPa) was applied by means of the spring loaded linear translation stage. In this mounting geometry, the signal from the negative side was typically found to be around 50% larger than the positive side, though this difference appears to decrease with increasing stress until the positive side exceeds the negative side. These changes are summarised in FIG. 7. Throughout all of the above tests, which involved several thousand separate measurements, no reduction in the signal amplitude for any of the mounting geometries used was observed. This establishes that the sensor is robust and reliable in spite of the mechanical clamping forces used to fix it in place and repeated cycling of applied tensile stress.

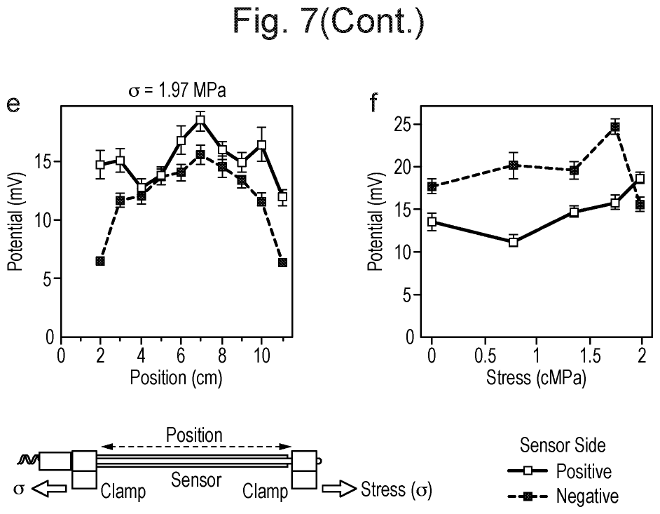

To determine if we could reduce the active sensor area and thereby lower potential production costs, the effect of reducing the sensor width on the measured signal amplitude was tested. The width of the PVDF was reduced, and the encapsulating tape was retained as shown in FIG. 8a. We observed a sharp decline in the measured potential in all three of the geometries measured (end clamped, one rib, and two ribs geometry), deviating from linearity likely due to presence of defects and cracks in the graphene film (FIGS. 8b to 8d). As these defects decrease the number of conduction pathways and therefore the measured potential, the effect becomes more significant when the width of the strip is reduced. The non-zero potential at zero strip width is a result of a small amount of charge generated by the remaining stub of PVDF in contact with the electrodes as a result of deflection of the encapsulating tape, despite the absence of PVDF in the sensor area. Due to the large decrease in signal magnitude and the instability in the signal along the strip as the width decreased, we proceeded with the initial strip width of 12 mm.

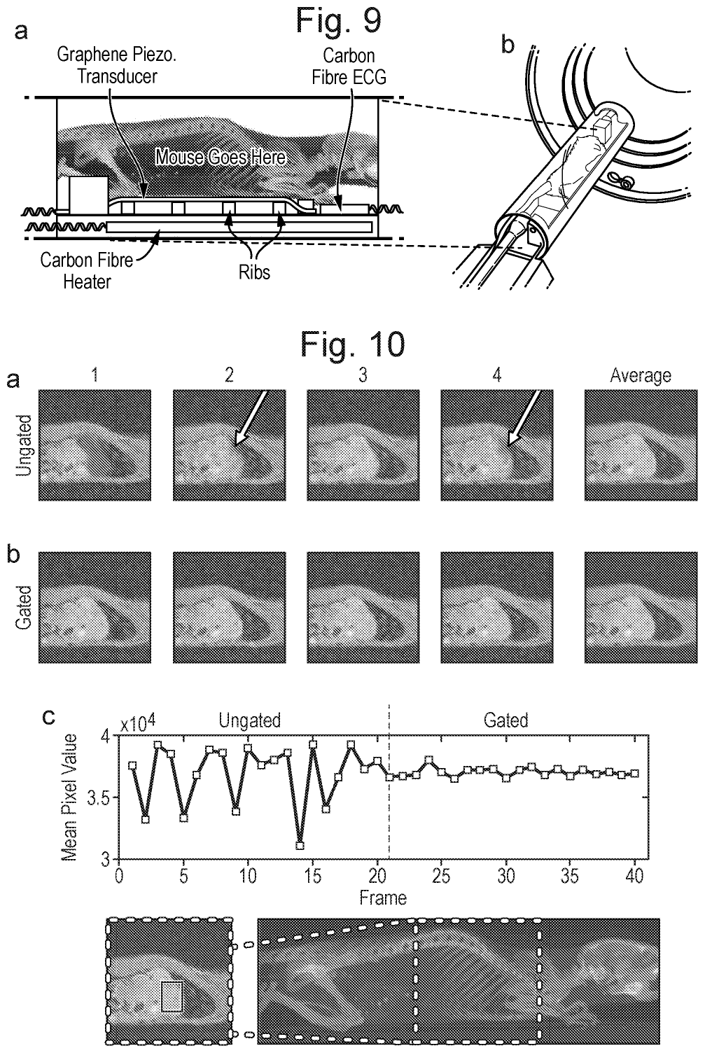

The final sensors were then integrated into the cradle. By compressing the sensor and securely clamping the ends and thus introducing a small positive curvature, we were able to ensure consistent contact with the animal while limiting the pickup of vibrations during scanning, with detection of the sensor enabled by several ribs along the length of the cradle. A schematic of the final cradle is shown in FIG. 10a, and an image of the cradle with animal in place prior to insertion into a CT scanner is shown in FIG. 10b. This cradle permits fully integrated MRI and CT compatible homeothermic maintenance and measurement of ECG and respiratory signals, the latter facilitated by the sensor of the invention, in a single user-friendly unit.

### Example 8—MRI Tests Performed Using Sensor

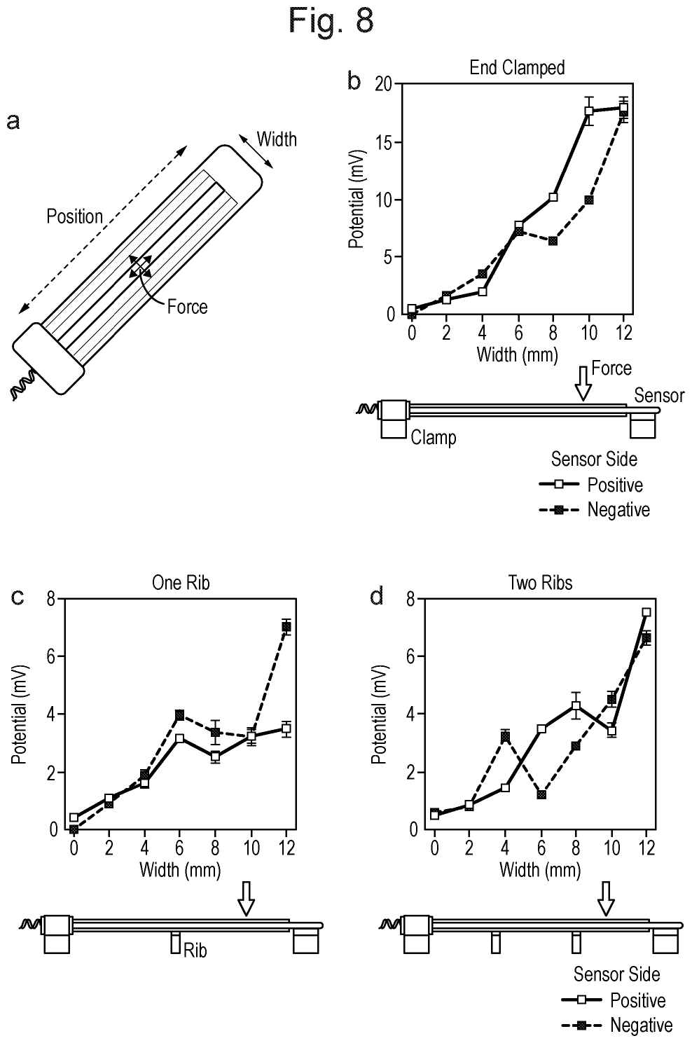

To verify the function of the final sensor in vivo, we performed MRI and CT imaging of live subjects using the measurement apparatus detailed in FIG. 9. MRI imaging was performed using a 7 T magnet, 210 VNMRS horizontal bore preclinical imaging system with 120 mm bore gradient insert (Varian Inc.). A 25 mm ID quadrature birdcage coil with 35 mm RF window length (Rapid Biomedical GmbH) was used for transmission and reception of RF signals. A constant TR, steady-stage respiration gated gradient echo imaging procedure was used for in vivo imaging. Typical parameters were TR=16.8 ms, TE=12 ms, FA=10°, THK=2 mm, in plane resolution=250 mm and image matrix=128×128.

We performed a dynamic scan without gating (FIG. 10a), and compared this to a scan using our device for respiratory gating of the image acquisition (FIG. 10b). Despite again being able to also measure cardiac signals with our device, due to complications with signal processing we elected only to demonstrate respiratory gating at this stage. The two signals could simply be used in tandem, or owing to their different rates could be separated by means of band pass filters.

With reference to the average gated scan the effect of respiration on the ungated image is clear, with significant time dependent movement visible across the sequential frames shown, in contrast to the good stability of the gated images. To show this graphically, we measured the mean pixel values across a cross-section in the area of the liver, and plotted this against time for both the ungated and gated scans in FIG. 10c, showing the marked increase in stability afforded by our device. This represents a near sevenfold reduction in the standard deviation of the mean pixel values in this area of greatest distortion, and a twofold reduction in the standard deviation of the overall image.

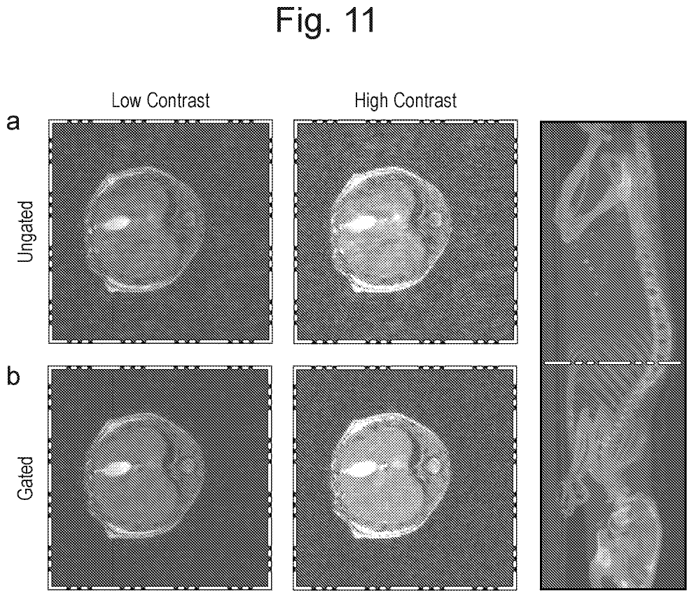

We subsequently performed high resolution acquisition with and without gating. These images serve to further highlight the impact of respiration on image quality: without gating, blurring around the lungs, liver, and surrounding tissues occurs, and significant artefacts can be observed around the body at higher contrast (FIG. 11a); with gating, the image is largely free of artefacts and streaking (FIG. 11b). The response of the graphene sensor was largely stable throughout MRI scanning, and we observed very little shift to the signals produced. We emphasise that while respiratory gating has been possible using a variety of techniques for several decades, our device enables this with, to the best of our knowledge, an unprecedented level of simplicity, signal stability, and flexibility to different systems, with clear benefits over existing technology to small animal imaging in terms of both throughput and user error minimisation.

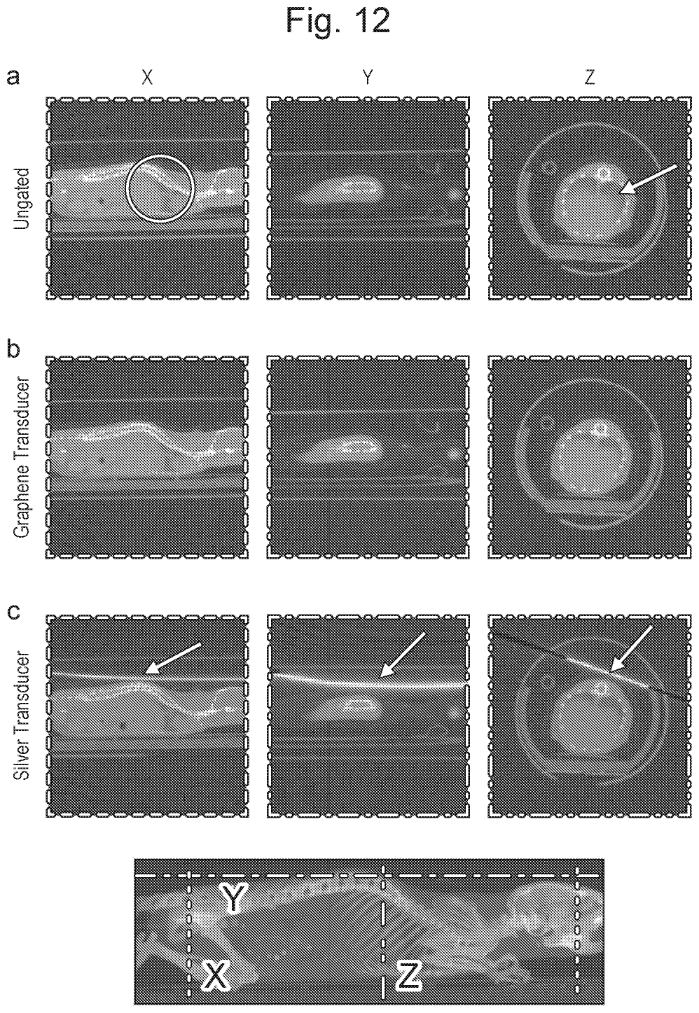

We further demonstrated the utility of the device to applications requiring coregistration by transferring the sedated animal and cradle to a CT scanner. By removing the need to repressurise and calibrate the respiratory balloon, the cradle can rapidly be reconnected to the new scanners monitoring systems, with minimum disturbance to anaesthesia and thermoregulatory systems. Body and sensor movement are minimised without the need to securely clamp the animal—reducing the risk of injury in contrast to respiratory balloons—by the secure mounting of the respiratory monitor over the majority of the bottom surface of the cradle. As a consequence, we observed very little change in the measured respiratory signal as a result of the transfer. In keeping with the MRI measurements, we performed an ungated scan (FIG. 12a), and compared this to a scan gated with the graphene contacted sensor (FIG. 12b). Note the blurring around the liver and the area of the thoracic cavity highlighted in the ungated images. The graphene transducer is visible as the grey line immediately beneath the animal.

Since its impact is much more noticeable and therefore more readily visualised than the detuning of the magnet and smaller artefacts in MRI, here we also demonstrate the effect of a silver contacted transducer on the reconstructed scan, with the bright streaked artefacts in FIG. 13c resulting from the strong X-ray scattering of the sensor. The silver contacted sensor was placed on top of the animal to avoid disturbing its position between scans. While the effect of respiration on the image quality is limited, our device simplifies cradle transfer without disturbing the animal's position with the result that the resolution of the co-registered image is enhanced. This versatility can be further extended to techniques requiring coregistration where the stability afforded by gating is more important, for example imaging techniques such as positron emission tomography, or treatment methods like radiotherapy. As in the simulated respiration measurements outlined in FIGS. 5 to 7, we observed no reduction in the sensitivity of the device in the >20 hours of in vivo testing.

1. A method of imaging a subject, wherein the method comprises:
(i) placing a sensor in contact with a subject, wherein the sensor comprises one or more piezoelectric materials and one or more electromagnetically transparent conductive materials; and
(ii) obtaining an image of a region of the subject with an imaging apparatus.

2. A method according to claim 1, wherein the method of imaging comprises a magnetic resonance imaging (MRI) method, a computed tomography (CT) method, or a positron-emission tomography (PET) method.

3. (canceled)

4. A method according to claim 1, wherein the one or more electromagnetically transparent conductive materials comprise (i) one or more electromagnetically transparent conductive nanomaterials such as a two-dimensional material, a one-dimensional material (e.g. a carbon nanotube film, or a metal nanowire film), a composite material comprising a two-dimensional material (e.g. graphene or niobium selenide) and one or more additional nanomaterials, or any combination thereof; (ii) one or more conductive polymers such as polyaniline, polyindole, polypyrrole, poly(3, 4-ethylenedioxythiophene) polystyrene sulfonate (PEDOT:PSS), or a combination thereof; or (iii) a film of electromagnetically transparent conductive particles, such as graphite, carbon black, or a combination thereof.

5. (canceled)

6. (canceled)

7. (canceled)

8. A method according to claim 1, wherein the one or more electromagnetically transparent conductive nanomaterials comprises graphene (e.g. graphene synthesised by chemical vapour deposition (CVD).

9. (canceled)

10. (canceled)

11. (canceled)

12. (canceled)

13. A method according to claim 1, wherein the one or more electromagnetically transparent conductive materials comprise a composite film, wherein the composite film comprises one or more electromagnetically transparent conductive nanomaterials, electromagnetically transparent conductive particles such as microparticles, or a combination thereof; and a polymer binder (e.g. polyaniline, polyindole, polypyrrole, poly(3, 4-ethylenedioxythiophene) polystyrene sulfonate (PEDOT:PSS), poly(methyl methacrylate), polystyrene, polycarbonate, acrylonitrile butadiene styrene, polyethylene, polypropylene, polyurethane, poly(lactic acid), poly(vinyl chloride), epoxy resins, polyamide, or a combination thereof).

14. (canceled)

15. (canceled)

16. A method according to claim 1, wherein the one or more electromagnetically transparent conductive materials comprises a film deposited from a liquid suspension of particles of the one or more electromagnetically transparent conductive materials, optionally wherein the particles of the one or more electromagnetically transparent conductive materials comprise graphene, graphite, or a combination thereof.

17. A method according to claim 1, wherein the one or more piezoelectric materials comprise one or more piezoelectric polymers (e.g. one or more fluorinated piezoelectric polymers), one or more piezoelectric polymer-composite materials, one or more composite materials comprising one or more polymers and one or more piezoelectric ceramic materials, or a combination thereof.

18. (canceled)

19. A method according to claim 17, wherein the one or more piezoelectric polymers or one or more piezoelectric polymer-composite materials comprise polyvinylidene fluoride (PVDF), a copolymer of polyvinylidene fluoride (PVDF) (e.g. polyvinylidene fluoride-trifluoroethylene-chlorotrifluoroethylene [P(VDF-TrFE-CTFE)], polyvinylidene fluoride-trifluoroethylene-chlorofluoroethylene [P(VDF-TrFE-CFE)], or a combination thereof), or a combination thereof.

20. (canceled)

21. A method according to claim 17, wherein the one or more piezoelectric polymer-composite materials comprise one or more nanomaterials such as graphene, carbon nanotubes, fullerenes, or any combination thereof; or wherein one or more composite materials comprising one or more polymers and one or more piezoelectric ceramic materials comprises a polymer selected from polyvinylidene fluoride (PVDF), polydimethylsiloxane, epoxy resin, polyurethane, or a combination thereof; and a ceramic selected from lead zirconate titanate, (PZT), lanthanum-modified lead zirconate titanate (PLZT), quartz, lithium niobate, or a combination thereof.

22. (canceled)

23. (canceled)

24. A method according to claim 1, wherein the sensor comprises a film of piezoelectric polymer (e.g. PVDF) with a film of graphene applied to the upper and lower surfaces of the piezoelectric polymer film, wherein the piezoelectric polymer film is from 9 μm to 750 μm in thickness.

25. (canceled)

26. (canceled)

27. A method according to claim 24, wherein the films of graphene are from 1 to 100 layers in thickness; optionally wherein each graphene film comprises a graphene monolayer.

28. (canceled)

29. A method according to claim 24, wherein the sensor further comprises a film of polymer such as polyethylene terephthalate, polypropylene, or polyethylene exterior to each film of graphene.

30. A method according to claim 24, wherein the sensor is from 5 mm to 20 mm in width; and/or wherein the sensor is from 50 mm to 150 mm in length.

31. (canceled)

32. A method according to claim 1, wherein the sensor further comprises a pair of electrodes (e.g. metal electrodes) that are electrically coupled with the one or more electromagnetically transparent conductive materials; wherein the electrodes are positioned in the sensor such that they do not interfere with the image of the region of the subject being obtained.

33. (canceled)

34. (canceled)

35. A method according to claim 1, wherein the subject is a mammalian subject, such as a human subject or a mammalian subject of the order Rodentia Ea rodent) (e.g. a mouse or rat).

36. (canceled)

37. A method according to claim 1, wherein the region of the subject comprises the entirety of the subject.

38. A method according to claim 1, wherein the method further comprises detecting movement of the subject with the sensor; wherein the movement of the subject is movement associated with the breathing of the subject and/or the heartbeat of the subject.

39. (canceled)

40. (canceled)

41. A method according to claim 38, wherein the method further comprises detecting movement of the subject with the sensor, wherein the movement of the subject is movement associated with the breathing of the subject or the heartbeat of the subject; and obtaining the image of the region of the subject at a time interval between breaths or heartbeats of the subject.

42. A sensor for use in an imaging method, wherein the sensor comprises a film comprising one or more piezoelectric materials and a film comprising one or more electromagnetically transparent conductive materials applied to the upper and lower surfaces of the film comprising the one or more piezoelectric materials.

43. (canceled)

44. An imaging system, wherein the system comprises an imaging apparatus such as an MRI scanner, a CT scanner, or a PET scanner, and a sensor according to claim 42.

45. (canceled)

46. (canceled)

47. (canceled)

48. (canceled)

49. (canceled)
